(19) United States
(12) Patent Application Publication
(10) Pub. No.: US 2008/0113008 A1
(43) Pub. Date: May 15, 2008

(54) Absorbent fabric implant

(76) Inventor: Karen Roche, Stillwater, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/901,237

(22) Filed: Sep. 14, 2007

Related U.S. Application Data
(60) Provisional application No. 60/844,473, filed on Sep. 14, 2006.

(51) Int. Cl. A61K 38/18; A61K 9/70; A61K 9/22

(52) U.S. Cl. 424/443; 604/890.1; 514/12

(57) ABSTRACT

The present invention is a method and device for treating a tissue defect, disease or abnormality. The device includes an absorbent container adapted to be placed at a tissue site, the container constructed and arranged to absorb a bioactive solution prior to the placement at the tissue site and to carry the solution to the tissue site such that the solution interacts with the tissue and wherein the absorbent container is substantially free of a metal support structure. The method includes providing an absorbent container substantially free of a metal support structure, soaking the container in a bioactive solution such that the solution is absorbed by the container and placing the container at the tissue site such that the solution interacts with the tissue.

### RELATED APPLICATION

The present application claims the benefit of U.S. Provisional Application No. 60/844,473 filed Sep. 14, 2006, which is incorporated herein in its entirety by reference.

### FIELD OF THE INVENTION

This invention relates to methods and devices for localized delivery of bioactive and/or pharmaceutical solutions. More particularly, the present invention relates to medical devices designed as absorbent carriers for bioactive solutions such as those containing antibiotics, bone morphogenic proteins, or pharmaceuticals appropriate to the treatment of a particular tissue defect, abnormality or disease, where the devices can be used to promote healing and/or deliver treatment.

### BACKGROUND OF THE INVENTION

Tissue abnormalities and defects arising from disease, trauma, or metabolic disorders can cause pain and in extreme cases can be disabling. For example, fractures, bony cysts, or tumors can be painful and debilitating. Similarly, low back pain, often arising from intervertebral disc degeneration even in the absence of obvious trauma or metabolic disorder, is one of the most common causes of disability for the middle-aged working population. In addition to the discomfort and pain experienced by the individual, substantial costs are borne by society, including costs for the diagnosis and treatment, and the cost of payments for disability benefits. Additionally, the lost job productivity can also be substantial.

One method for treating low back pain involves stabilizing a spinal motion segment by inserting a fabric bag into an opening formed in the intervertebral disc space. The disc space can then be expanded to a desired position by introducing fill material into the interior of the fabric bag to stabilize the vertebral bodies that define a motion segment and thereby reduce pain associated with the motion segment. The fabric bag can be porous, which can facilitate the ingrowth of bony trabeculae and/or fibrous elements into and through the fabric bag, thereby further stabilizing the motion segment and relieving back pain. The fill material selected can be one which supports bone growth or which provides other desirable mechanical properties such as the ability to provide compressive strength and stability while permitting some limited motion. Methods and devices for stabilizing a spinal motion segment in this manner are further described in, for example, U.S. Pat. No. 5,549,679, entitled “Expandable Fabric Implant For Stabilizing The Spinal Motion Segment.”

Bony defects can often be successfully treated with the implantation of appropriate bone graft or graft substitute materials which can help to restore bony alignment and provide sufficient mechanical stability to reduce pain and improve function. Such materials can be most effective for providing mechanical support when they are contained, such as within an implantable porous container. The container can be a fabric bag, such as is described in co-pending U.S. patent application Ser. No. 10/440,036, which is inserted into the defect space and then filled with graft or similar materials. Filling the fabric bag with appropriate pressure reduces the defect, thus restoring anatomic alignment to the defect site, and can also provide an osteoconductive scaffold to support future bony ingrowth and graft incorporation.

While providing an osteoconductive scaffold helps to promote new bone growth, it is desirable to provide additional therapeutic solutions to the host tissue site. Conventional methods for delivering therapeutic solutions to host tissue sites include injections and systemic delivery of solution. While delivery of therapeutic solutions by injection can localize the solution to a particular treatment area, the treatment will be temporary and can not last for longer than it takes the body to metabolize the solution. Systemic delivery, on the other hand, has no ability to localize the delivery of the therapeutic solution to a particular treatment site.

Another conventional method to deliver a therapeutic agent to a treatment site is to soak an absorbable collagen sponge in a therapeutic solution. The collagen sponge is then implanted at the host treatment site. Using a collagen sponge as the carrier for bioactive solutions has several disadvantages. It can be difficult to engineer the collagen sponges with the desired properties. Further, because bovine collagen is generally used to manufacture the collagen sponges, the sponges may produce an undesired immune response in the human host tissue. Also, collagen sponges do not effectively bear compressive loads and thus, in interbody spinal applications, for example, the collagen sponges must usually be placed inside a spinal cage or other load bearing structure. Finally, the collagen sponge takes up space in the anatomic defect and when used with an implant, the sponge may alter the mechanical characteristics of the implant construct.

U.S. Pat. No. 6,827,743, entitled “Woven Orthopedic Implants,” describes an orthopedic implant made from a metallic mesh material that may have alternating strands of metal wires and collagen, the collagen strands of the mesh members may be soaked in recombinant human bone morphogenic protein. While this approach solves some of the problems of conventional collagen sponges, the woven metallic mesh implant has certain disadvantages. Because the mesh implant is comprised of alternating strands of metal wires and collagen, at least half of the mesh fibers are substantially nonporous metal wires and thus the volume of recombinant human bone morphogenic protein that could be absorbed by the device is minimal. Further, the collagen strands used in the metallic mesh construct may still cause an immune response just as is the case with conventional collagen sponges. Finally, any surface treatment to the metallic mesh must be done during the manufacturing process.

Generally, medical devices designed to treat, stabilize or repair bony defects do not carry medicinal substances that may further the local healing by preventing infection, such as, for example, antibiotics, or by promoting the growth of bone tissue such as, for example, bone morphogenic proteins. In the case of bony defects caused by tumor, or the necessity to remove a tumor, medical devices designed to stabilize the resulting defect generally do not carry pharmaceutical agents which might help to prevent regrowth or spread of the tumor itself. There is a need for a medical implant that carries and retains bioactive and/or pharmaceutical solutions for localized delivery of the therapeutic solution such that the implant can bear compressive loads and be implanted in a minimally invasive manner.

### SUMMARY OF THE INVENTION

In a first aspect, the invention pertains to an absorbent medical device comprising a container having a wall membrane, the wall membrane defining an interior and an exterior of the container, wherein at least a portion of the wall membrane may be specially fabricated so as to imbibe a bioactive solution such as one containing an antibiotic, a bone morphogenic protein, a pharmaceutical compound or any other solution appropriate to the treatment of the tissue defect, disease or abnormality. In some embodiments, the wall membrane can be flexible and/or porous.

In another aspect, the invention pertains to an absorbent medical device comprising a flexible container having a wall membrane, the wall membrane defining an interior and an exterior of the container, the wall having at least one passage connecting the interior with the exterior, adapted to permit material to be introduced into the interior of the container, wherein at least a portion of the wall membrane may be specially fabricated so as to imbibe a bioactive or medicinal solution such as one containing an antibiotic, a bone morphogenic protein, or a pharmaceutical compound appropriate to the treatment of the disease or damage.

In a further aspect, the invention pertains to an implantable device comprising an expandable bag formed of a porous fabric wall that includes a plurality of openings between about 0.25 to about 5.0 mm in diameter. In these embodiments, the bag can further include an opening through which the bag may be filled. Additionally, at least a portion of the outside surface of the bag may be specially fabricated so as to imbibe a bioactive solution such as one containing an antibiotic, a bone morphogenic protein, a pharmaceutical compound, or combinations thereof, appropriate to the treatment of the bony defect or abnormality. In some embodiments, the bag can be formed from a fabric, wherein the fabric can absorb a volume of bioactive or medicinal solution that is greater than the volume of the fabric.

In another aspect, the implantable fabric container of the invention may be knitted or woven from fibers which are processed to increase their physical bulk and/or effective surface area so as to enhance their ability to absorb and to retain solutions in which the fabric container is immersed. Such fibers can be knitted or woven using the same type of textile manufacturing processes as for non-bulked (relatively smooth) fibers. In some embodiments, the fibers can be processed so that the effective surface area of the fibers is from about 1.5 times to 6 times the surface area of untreated fibers. In other embodiments, the fibers can be processed so that the effective surface area of the fibers is from about 2 times to about 5 times the surface area of untreated fibers. Preferably, the bulked fibers retain increased bulk until the fibers experience tension and are pulled taut. In the case of an expandable porous fabric container, this feature is advantageous because the collapsed fabric container may be soaked in solutions and the bulked fibers will absorb a greater amount of solution than a fabric container made of non-bulked fibers, but because the bulking diminishes upon the expansion of the fabric container, the size of the pores will not be adversely affected by the bulked fibers.

In a further aspect, the invention pertains to an apparatus comprising an absorbent medical device and a package for storing the medical device, the package defining an interior space isolated from the ambient atmosphere. In some embodiments, the interior space of the package can contain the medical device presoaked in a solution containing the desired bioactive or pharmaceutical solution. In other embodiments, the interior space of the package can include the medical device with a solution containing the desired bioactive or pharmaceutical solution in a separate container packaged together with or separate from the medical device.

In another aspect, the invention pertains to a method of treating a bony defect or abnormality that positions an implantable device into the bony defect or abnormality, the implantable device comprising a flexible container having a wall membrane, the wall membrane defining an interior and an exterior of the container, wherein at least a portion of the wall membrane may be specially constructed so as to imbibe a bioactive solution such as one containing an antibiotic, a bone morphogenic protein, or a pharmaceutical compound appropriate to the treatment of the bony defect or abnormality. In this embodiment, the absorbent implantable device is immersed in a bioactive solution such as one containing an antibiotic, a bone morphogenic protein, a pharmaceutical compound, or combinations thereof, appropriate to the treatment of the bony defect or abnormality prior to its implantation. Insertion and deployment of the implantable flexible container ensures delivery of the bioactive solution to the specific implant site.

Subsequent filling of the at least a portion of the interior of the flexible container with fill material such as, for example, bone graft or bone graft substitute material as described in, U.S. Pat. Nos. 5,549,679 and 7,025,771 and U.S. patent application Ser. No. 10/440,036, the entirety of which are hereby incorporated by reference, expands the container into a relatively rigid composite construct. This rigidity allows the filled fabric container to bear compressive loads while delivering local site specific therapeutic solutions. Further, the process of filling the container to create a relatively rigid construct induces tension on the fibers. This tension diminishes the fiber's bulk and maximizes the effective pore size of the implant. As the tension diminishes the fiber's bulk, a portion of the bioactive solution is expressed out of the fibers, thereby delivering the imbibed solution into the tissues immediately surrounding the implant

In a further aspect, the invention pertains to a method of forming an implantable device comprising soaking an absorbent implantable device in a bioactive solution just prior to implantation of the device. In one embodiment, the device is soaked in a solution comprising bone morphogenic proteins or other bioactive/pharmaceutical substances for about 2 to about 140 minutes.

In another embodiment, the present invention relates to a kit comprising a tray having a bioactive solution, an absorbent implantable device, and a package adapted to isolate and store the tray and the absorbent implantable device. In these embodiments, the bioactive solution can comprise an antibiotic, a bone morphogenic protein, a pharmaceutical compound, or combinations thereof, and the absorbent implantable device can comprising an expandable bag formed of a porous fabric wall that includes a plurality of openings into the interior of the container between about 0.25 to about 5.0 mm in diameter.

### BRIEF DESCRIPTION OF THE FIGURES

FIG. 1 is a top plan view of an expandable bag having a porous fabric wall with a plurality of openings, wherein at least a portion of the outside surface of the fabric wall may imbibe a bioactive solution for the treatment of a tissue defect, disease or abnormality in accordance with one embodiment of the present invention.

FIG. 2 is a side elevational view of the expandable bag of FIG. 1.

FIG. 3 depicts fibers which have been processed to increase their physical bulk in accordance with one embodiment of the present invention.

### DETAILED DESCRIPTION OF THE INVENTION

Improved medical devices for treating tissue defects, disease or abnormalities can comprise one or more absorbent surfaces which may imbibe a bioactive solution such as one containing an antibiotic, a pharmaceutical agent, or a bone morphogenic protein such as, for example, recombinant human bone morphogenetic protein (rhBMP). The device of the present invention carries and retains the desired solution directly to the host tissue site. Due to the local and site-specific presence of the imbibed solution, the improved medical devices can interact with the damaged or diseased tissue. This site-specific presence provides an advantage over injected or systemically delivered therapeutic solutions, which may rapidly metabolize resulting in a diminished therapeutic effect. The medical devices of the present invention can be implantable devices. In some embodiments, the medical device can be an apparatus designed to stabilize a spinal motion segment comprising a container having a wall membrane, the wall membrane defining an interior and an exterior of the container.

In some embodiments, a portion or all of the wall membrane of the container can be formed from one or more porous materials that can imbibe a bioactive solution. Additionally, the wall membrane can be also be porous such that desired materials may move into and out of the interior of the container. In the case of the treatment of bony abnormalities, the porous wall membrane can facilitate bony in-growth or interdigitation between the surrounding bone and the implantable device. In this embodiment, the wall membrane may be made up of, for example, a plurality of porous woven fibers, wherein the plurality of porous woven fibers can further define a plurality of pores through the wall membrane into the interior of the container. The pores though the wall membrane can be formed by the relative orientation or packing of adjacent fibers. Presence of imbibed growth factors such as bone morphogenic proteins can aid in the treatment of bony abnormalities by their ability to promote the generation of new bone tissue to facilitate the stabilizing interdigitation between host bone and osteoconductive materials, such as bone graft or bone graft substitutes which may be introduced into and contained by the porous implantable device.

Generally, the wall membrane can be formed from one or more fabric materials. The use of term “fabric” herein is meant to include the usual definition of that term and to include any material that functions like a fabric, i.e., materials having suitable flexibility and porosity. In some embodiments, the wall membrane can be formed from a fabric material and can be substantially free of a metal support structure(s). The term “substantially free of a metal support structure” is being used to indicate that the wall membranes can comprise less than 30%, preferably less than 15% and more preferably less than 5% metal support structures such as metal wires, bands and the like.

The medical devices of the present invention are devices designed to repair and/or stabilize damaged and/or diseased tissue, which may include bony defects or other structural and metabolic abnormalities, wherein desired portions of the medical device can imbibe, or comprise, a bioactive substance. Providing absorbent medical devices can facilitate delivery of bioactive substances to desired host tissue, which may help to variously prevent infection, when antibiotics have been imbibed; to promote the growth of new tissue and decrease healing time and/or increase healing rate, when growth factors such as bone morphogenic proteins have been imbibed; or to provide other locally beneficial pharmaceutical action, such as when a chemotherapeutic solution has been imbibed. The medical devices may be manufactured to absorb over time into the host tissue and in the case of bony defects aide in the remodeling of the bone.

In embodiments where the medical device comprises a porous or absorbent surface, the porous or absorbent surface can be exposed to a solution comprising a bioactive substance in solution such that the solution can absorb into the surface of the device. The physical bulk of the fibers creating the absorbent surface may also aid in retaining or entrapping the proteins or bioactive molecules which are dissolved within the solution. The bulkiness of the fibers can increase the effective surface area of the fibers and can create a mechanically tortuous surface which may enhance the retention and allow a delay in metabolization or dispersion of the medicinal agent delivered, thus enhancing its effectiveness. Therapeutic solutions are often rapidly metabolized and thus solutions that are injected locally or systemically delivered to the treatment site may not remain at the site absent an absorbent carrier that can retain the solutions for an effective period of time. The ability of the bulked fibers to carry and retain the bioactive and/or pharmaceutical solutions such that the solutions may be gradually released over time thus provides an advantage over locally or systemically delivered solutions.

In other embodiments, a porous substrate such as a fiber or the like can be attached to desired surfaces of a medical device to facilitate the ability of the medical device to imbibe a bioactive solution intended to enhance treatment of the implantation site. As described above, the medical devices of the present disclosure can comprise at least one surface designed to imbibe bioactive substances which can facilitate the treatment of a tissue defect, disease or abnormality. In general, the medical devices can be any device designed to treat, heal, stabilize and/or fixate damaged or diseased tissue or skeletal abnormalities.

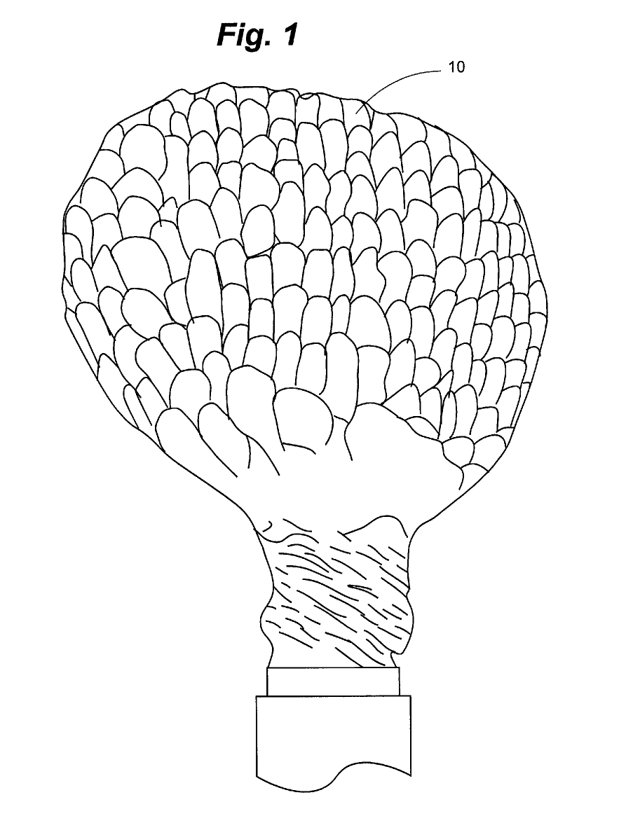

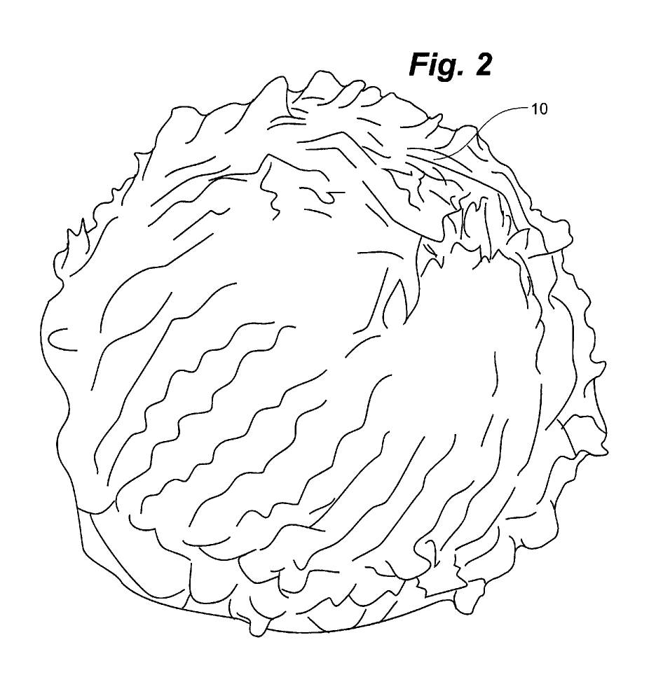

As depicted in FIGS. 1 and 2, in some embodiments, the medical device can comprise an expandable bag 40 optionally having a fill opening 48 that permits fill material to be introduced into the interior of expandable bag. In some embodiments, expandable bag 40 can be formed from a fabric that is woven or form-molded to a density that allows the ingrowth and/or through growth of blood vessels, fibrous tissue and/or bony trabeculae. Additionally, in some embodiments, the density of the fabric can be selected to permit at least the liquid fill material to flow out of the interior of the bag into the surrounding tissue. The fill opening of the bag may be closed, sealed or in some embodiments the fill opening may be left open.

Additionally, the fibers from which the fabric is manufactured can be specially processed so as to increase their physical bulk and enhance their ability to imbibe solutions. In some embodiments, the pores of the fabric into the interior of the container, when expanded, can have a diameter from about 0.25 to about 5.0 mm to permit the ingrowth or through growth of blood vessels. Suitable expandable bags are described further in U.S. Pat. No. 5,549,679 to Kuslich, entitled “Expandable Fabric Implant for Stabilizing the Spinal Motion Segment,” and U.S. Pat. No. 6,712,853 entitled, “Annulus Reinforcing Band,” U.S. patent application Ser. No. 10/440,036 to Kuslich et al., entitled “Expandable Porous Mesh Bag Device and Methods of Use for Reduction, Filling, Fixation, and Supporting of Bone,” and U.S. patent application Ser. No. 10/804,761 to Hochschuler et al., entitled, “Method and Apparatus for Treating a Vertebral Body,” all of which are hereby incorporated by reference herein.

In one embodiment, to prepare the medical devices of the present disclosure a suitable medical device such as, for example, expandable bag 40 can be soaked in a solution containing a growth factor such as a recombinant human bone morphogenic protein, rhBMP-2. Soaking expandable bag 40 in a solution comprising rhBMP-2 can facilitate absorption of the rhBMP-2 into the fabric material of bag 40 such that bag 40 forms a targeted delivery vehicle for rhBMP-2. In some embodiments, the medical device can be soaked in a solution comprising, for example, from about 0.005 mg/ml to about 0.040 mg/ml rhBMP-2. One of ordinary skill in the art will recognize that additional ranges of rhBMP-2 concentration within these explicit ranges are contemplated and are within the scope of the present disclosure. Superconcentrated solutions may be prepared which would increase the effective dosage of the agent being delivered by the device. In some embodiments, the medical devices can be soaked in a solution comprising rhBMP-2 from about 2 to about 140 minutes. One of ordinary skill in the art will recognize that additional ranges of soak time within these explicit ranges are contemplated and are within the scope of the present disclosure.

In embodiments where the medical devices comprise a fiber or absorbent material that can absorb a solution, the medical devices can be soaked in a bioactive and/or pharmaceutical solution such that the volume of solution absorbed by the fiber or absorbent material is from about 1 to about 5 times the volume of the fiber or absorbent material. For example, a medical device such as expandable bag 40 formed from 0.2 cc of fiber material can be soaked in a solution until the fiber material has absorbed from about 0.25 cc to about 1.0 cc of the solution.

In some embodiments, the medical devices can be packaged in a package, which can facilitate storage and/or transport of the medical devices. Generally, the packages can define an interior space that is isolated from the ambient atmosphere and comprise a sterile solution. The package can be formed out of any suitable material for use in medical device applications including polymers, metals, metal alloys and combinations thereof. Suitable polymers include, for example, polyethylene (PE), polypropylene (PP), poly(tetrafluoroethylene) (PTFE), polycarbonates, polyurethanes, and blends and copolymers thereof. In some embodiments, the package can be filled with a particular gaseous compound, such as nitrogen or carbon dioxide. In other embodiments, the interior space of the package can be maintained in a vacuum or partial vacuum state.

In some embodiments, the package can be a polymeric bag that seals and isolates the medical device from the ambient atmosphere. In these embodiments, polymeric bag may contain a perforation formed into surface of polymeric bag, which allows a physician or other user to tear along a perforation to open the bag and access the enclosed medical device. In some embodiments, the interior space of the polymeric bag can comprise a medical device and a solution in contact with the medical device. The solution can comprise a solution of sterile saline or water containing a suitable bioactive agent such as antibiotic compounds, growth factors including bone morphogenic proteins, or other Medicinal/pharmaceutical agents. In some embodiments, the saline solution can comprise an aqueous solution having from about 0.5% to about 2.0% by weight sodium chloride.

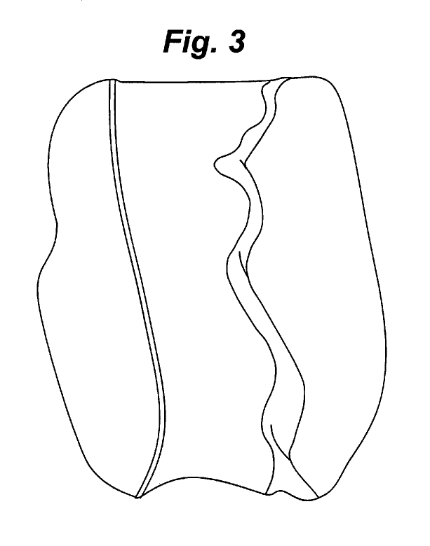

As depicted in FIG. 3, the properties of a conventional fiber 3a can be altered to increase the effective surface area and/or the physical bulk of the fibers as in 3b to enhance the ability of the fiber to absorb solutions and to aid simple mechanical retention of the solute components (such as certain protein molecules) within the tortuous path of the altered fibers. In some embodiments, the bulked fibers can have an increased surface area from about 1.5 times to about 6 times the surface area of an untreated fiber, while in other embodiments the bulked fibers can have an increased surface area from about 2 times to about 5 times the surface area of an untreated fiber.

During use, desired surfaces of a medical device can be coated with an appropriate bioactive solution. In some embodiments, the medical device can be pre-treated with such a solution and stored in a package as described above. In other embodiments, the medical device can be soaked in a bioactive and/or pharmaceutical solution at the point of use, just prior to use of the medical device. The medical device can then be implanted or attached to a patient such that desired portions of the medical device are positioned in contact with or proximate to the bony defect or abnormality surface. Once positioned, the medicinal solution on the medical device is delivered to the interface between the device and the host tissue, where it can help to facilitate healing and/or stabilization of diseased and/or damaged tissue, or to protect against infection of the host tissue site.

The embodiments above are intended to be illustrative and not limiting. Additional embodiments are within the claims. Although the present invention has been described with reference to particular embodiments, workers skilled in the art will recognize that changes may be made in form and detail without departing from the spirit and scope of the invention.

What is claimed is:

1. A device for treating a tissue defect, disease or abnormality comprising:
an absorbent container adapted to be placed at a tissue site;
the container constructed and arranged to absorb a bioactive solution prior to the placement at the tissue site and to carry the solution to the tissue site such that the solution interacts with the tissue; and
wherein the absorbent container is substantially free of a metal support structure.

2. The container of claim 1, wherein the bioactive solution is selected from the group consisting of at least one of: an antibiotic, a bone morphogenic protein, a pharmaceutical, and any combination thereof.

3. The container of claim 1 including a fill opening adapted to permit the introduction of fill material into the container.

4. The container of claim 1, wherein the container is formed from one or more fabric materials.

5. The container of claim 4, wherein the fabric is processed to increase the fabric's physical bulk.

6. A device for treating a tissue defect, disease or abnormality comprising:
an absorbent container adapted to be placed at a tissue site;
the container constructed and arranged to absorb a bioactive solution prior to the placement at the tissue site;
the absorbent container being substantially free of a metal support structure; and
wherein the container includes a fill opening adapted to permit the introduction of fill material into the container.

7. The device of claim 6, wherein the bioactive solution is selected from the group consisting of at least one of: an antibiotic, a bone morphogenic protein, a pharmaceutical, and any combination thereof.

8. The container of claim 6, wherein the container is formed from one or more fabric materials.

9. The container of claim 8, wherein the fabric is processed to increase the fabric's physical bulk.

10. The container of claim 6, wherein the container is porous.

11. The container of claim 8, wherein the container is constructed and arranged such that the introduction of fill material induces tension on the fabric such that the bioactive solution is expressed out of the fibers into the tissue.

12. A method of treating a tissue defect, disease or abnormality including:
providing an absorbent container substantially free of a metal support structure;
soaking the container in a bioactive solution such that the solution is absorbed by the container;
placing the container at the tissue site such that the solution interacts with the tissue.

13. The method of claim 12 further including filling the container with fill material.

14. A method of treating a tissue defect, disease or abnormality including:
providing an absorbent container substantially free of a metal support structure;
providing a bioactive solution;
providing instructions including:
soaking the absorbent container in the bioactive solution;
placing the soaked container at a tissue site; and
filling the container with fill material.
